(19) United States
(12) Patent Application Publication
(10) Pub. No.: US 2004/0054386 A1
(43) Pub. Date: Mar. 18, 2004

(54) Device for the treatment of muscle or joint pain

(76) Inventors: Todd Martin, Dodgeville, WI (US); Mark Straubhaar, Barneveld, WI (US); Ronald W. Ignatius, Barneveld, WI (US)

Correspondence Address: MICHAEL BEST & FRIEDRICH, LLP, 100 E WISCONSIN AVENUE, MILWAUKEE, WI, 53202, US

(21) Appl. No.: 10/653,403

(22) Filed: Sep. 2, 2003

Related U.S. Application Data
(60) Provisional application No. 60/408,216, filed on Sep. 4, 2002.

(51) Int. Cl. A61N 1/08

(52) U.S. Cl. 607/88; 607/46

(57) ABSTRACT

Apparatus is provided for the treatment of a medical condition, such as muscle or joint pain. One embodiment of the apparatus is a hand-held device including a housing and at least one optoelectronic device, such as a light-emitting diode (LED), coupled to the housing. The optoelectronic device may be cooled by a cooling system. The cooling system may include a heat sink and a temperature sensor.

### RELATED APPLICATIONS

This application claims priority under 35 U.S.C. §119 to U.S. Provisional Patent Application No. 60/408,216 filed Sep. 4, 2002.

This invention was made with U.S. Government support under Contract DAAH01-03-C-R-120 awarded by the Defense Advanced Research Projects Agency (DARPA). The U.S. Government has certain rights in this invention.

### BACKGROUND OF THE INVENTION

This invention relates to a device for the treatment of muscle or joint pain. The device includes arrays of optoelectronic devices, such as light emitting diodes, that emit radiation suitable for the treatment of muscle or joint pain.

Biostimulation is a method of using monochromatic light to deliver photons to cytochromes in the mitochondria of cells. Cytochromes are light-sensitive organelles that act as an electron transport chain, converting energy derived from the oxidation of glucose into adenosine triphosphate (ATP)—the mitochondria's fuel. By directly stimulating cytochromes with monochromatic light, it is believed that more fuel is pumped into the mitochondria of cells, increasing the energy available to the cells. Increasing the energy available to the cell is believed to help relieve pain.

By pumping more fuel into the mitochondria, biostimulation is believed to increase the respiratory metabolism of many types of cells. The monochromatic light provided by biostimulation is believed to be absorbed by the mitochondria of many types of cells where it stimulates energy metabolism in muscle and bone, as well as skin and subcutaneous tissue. Specifically, biostimulation is believed to result in fibroblast proliferation, attachment and synthesis of collagen, procollagen synthesis, macrophage stimulation, a greater rate of extracellular matrix production, and growth factor production. Specifically, the growth factors that are produced include keratinocyte growth factor (KGF), transforming growth factor (TGF), and platelet-derived growth factor (PDGF).

One method of providing biostimulation is the use of lasers. Lasers can provide monochromatic light for the stimulation of tissues resulting in increased cellular activity during the healing process. Specifically, these activities are believed to include fibroblast proliferation, growth factor synthesis, collagen production, and angiogenesis.

Using lasers to provide monochromatic light for biostimulation has several disadvantages. First, lasers are limited by their wavelength capabilities. Specifically, the combined wavelengths of light optimal for treating muscle and joint pain cannot be efficiently produced, because laser conversion to near-infrared wavelengths is inherently costly. Second, lasers are limited by their beam width. A limited beam width results in limitations in the area which may be treated by lasers. Third, and most importantly, along with the production of monochromatic light, lasers produce a significant amount of heat. As a result of the production of heat, lasers cannot be used for extended treatment times or in applications in which the patient cannot tolerate heat.

### SUMMARY OF THE INVENTION

The invention provides a device for treating a medical condition, such as muscle or joint pain, using an array of optoelectronic devices, such as light-emitting diodes (LEDs). In one embodiment of the invention, a device for treating muscle or joint pain is a self-contained, self-powered, hand-held device that can emit radiation having a light intensity of at least approximately 30 milliwatts per centimeter squared. The device includes a housing, a portable power source disposed in the housing, and one or more optoelectronic devices disposed in the housing and coupled to the portable power source. The device also includes a cooling system disposed in the housing. The cooling system can dissipate the heat generated by the optoelectronic devices.

According to one embodiment of the method of the invention, a user positions a housing including optoelectronic devices adjacent to a muscle and/or ajoint of a patient. The user irradiates the muscle and/or the joint with radiation emitted by the optoelectronic devices. The emitted radiation has a wavelength suitable for the treatment of muscle and/or joint pain. The heat produced by the optoelectronic devices is dissipated through the housing.

According to another embodiment of the method of the invention, a user positions a housing adjacent to at least one of a muscle and a joint of a patient. A plurality of optoelectronic devices are disposed in the housing. The user irradiates the muscle and/or the joint with radiation emitted by the plurality of optoelectronic devices for a treatment session having a first duration. The plurality of optoelectronic devices are allowed to dissipate heat for a cooling-down period having a second duration, and the plurality of optoelectronic devices are prevented from emitting radiation during the cooling-down period.

### BRIEF DESCRIPTION OF THE DRAWINGS

These and other features of the present invention will be apparent to those skilled in the art from the following description of the preferred embodiments and the drawings, in which:

FIG. 1 is a top perspective view of a hand-held device according to one embodiment of the present invention.

FIG. 2 is a bottom perspective view of the hand-held device of FIG. 1.

FIG. 3 is a side elevational view of the hand-held device of FIG. 1.

FIG. 4 is a side elevational view of the hand-held device of FIG. 1 with a power source compartment cover removed.

FIG. 5 is an exploded side elevational view of the hand-held device of FIG. 1.

FIG. 6 is a perspective view of a heat sink, a circuit board, and a ceramic assembly of the hand-held device of FIG. 1.

FIG. 7 is a side elevational view of the heat sink, the circuit board, and the ceramic assembly of FIG. 6.

FIG. 8 is a side elevational view of the heat sink and the ceramic assembly of FIG. 6.

FIG. 9 is a side elevational view of the heat sink and the circuit board of FIG. 6.

FIG. 10 is a schematic diagram of a control circuit for use with the hand-held device of FIG. 1.

FIG. 11 is a current source module of the control circuit of FIG. 10.

FIG. 12 is a voltage reference module of the control circuit of FIG. 10.

FIG. 13 is a power control module of the control circuit of FIG. 10.

FIG. 14 is a power-on reset module of the control circuit of FIG. 10.

FIG. 15 is a temperature sensing module of the control circuit of FIG. 10.

FIG. 16 is a battery voltage sensing module of the control circuit of FIG. 10.

### DETAILED DESCRIPTION

In each of the embodiments of the present invention, at least one optoelectronic device is used to emit radiation for the treatment of a medical condition, such as for the treatment or relief of muscle or joint pain. The optoelectronic devices can be substantially monochromatic, double-heterojunction, Gallium-Aluminum-Arsenide (GaAlAs) LEDs of the type manufactured by Showa Denkoa or Stanley, both of Japan, or by Hewlett-Packard of Palo Alto, Calif. In some embodiments, the optoelectronic devices are connected together in a manner described in U.S. Pat. No. 5,278,432 issued Jan. 11, 1994 to Ignatius et al., which is incorporated herein by reference.

In some embodiments, the LEDs emit radiation at approximately 670 nanometers (nm)±approximately 15 nm, which is believed to be an optimal wavelength for relieving and potentially treating muscle and/or joint pain. Some embodiments of the invention include an array of LEDs that emit radiation. Other wavelengths may also be suitable for relieving and treating muscle and/or joint pain or for treating other medical conditions, such as approximately 300 nm to 950 nm, and more specifically, approximately 640 nm to 700 nm. Moreover, as further research is conducted, other wavelengths may be found to be effective. However, the present invention is not limited to the use of any specific wavelength. In some embodiments, the LEDs are wavelength specific in that the LEDs emit a certain wavelength when provided with power. For example, one or more wavelength-specific LEDs emitting radiation at 670 nm can be assembled onto a circuit board or any other suitable substrate in order to provide a hand-held device 10 that emits radiation at a central wavelength of 670 nm.

In addition to the wavelength of the radiation emitted by the LEDs, the following parameters should be considered to optimize the stimulative effect of the LEDs on biological tissues: the energy density required for activation (E/a)_{act}, the light intensity Iₛₜᵢₘ, and the total irradiation time Δtₜₒₜ. The parameters are interrelated according to the following equation,

(E/a)_{act}=Iₛₜᵢₘ×Δtₜₒₜ

where intensities necessary for stimulation Iₛₜᵢₘ should surpass a threshold intensity Iₒ, i.e.,

Iₛₜᵢₘ≧Iₒ.

Light intensities lower than threshold values Iₒ typically may not produce biostimulatory effects, even under prolonged irradiation times Δtₜₒₜ.

It is believed that the optimal energy densities for cellular activation (E/a)_{act} are approximately 4 to 8 Joules per centimeter squared. The light intensity (Iₛₜᵢₘ) of the radiation emitted by the LEDs may be approximately 30 to 80 milliwatts (mW) per centimeter squared, and up to approximately 200 milliwatts per centimeter squared. In one embodiment, the LEDs emit radiation at an intensity of approximately 50-60 milliwatts per centimeter squared. In some embodiments, the irradiation time Δtₜₒₜ per treatment period is about 88 seconds ±8 seconds.

In some embodiments, the LEDs emit radiation having a relatively constant light intensity over a treatment area. In one embodiment, the light intensity varies by less than about 30% over a treatment area of approximately ten square centimeters. For example, 4.8 LEDs per centimeter squared for a total of 48 LEDs can provide a relatively constant light intensity over a treatment area of approximately ten square centimeters. However, fewer than 4.8 LEDs per centimeter squared can be used if the LEDs emit radiation at a higher light intensity.

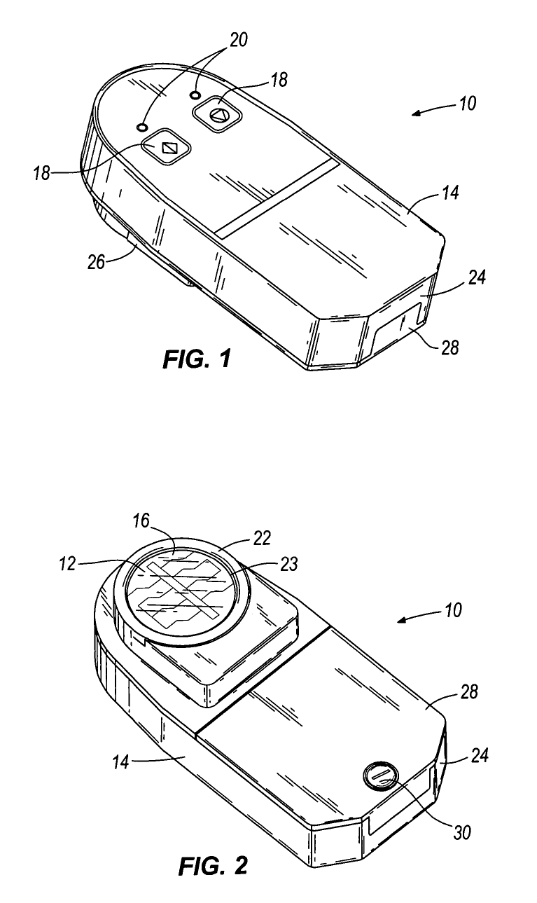

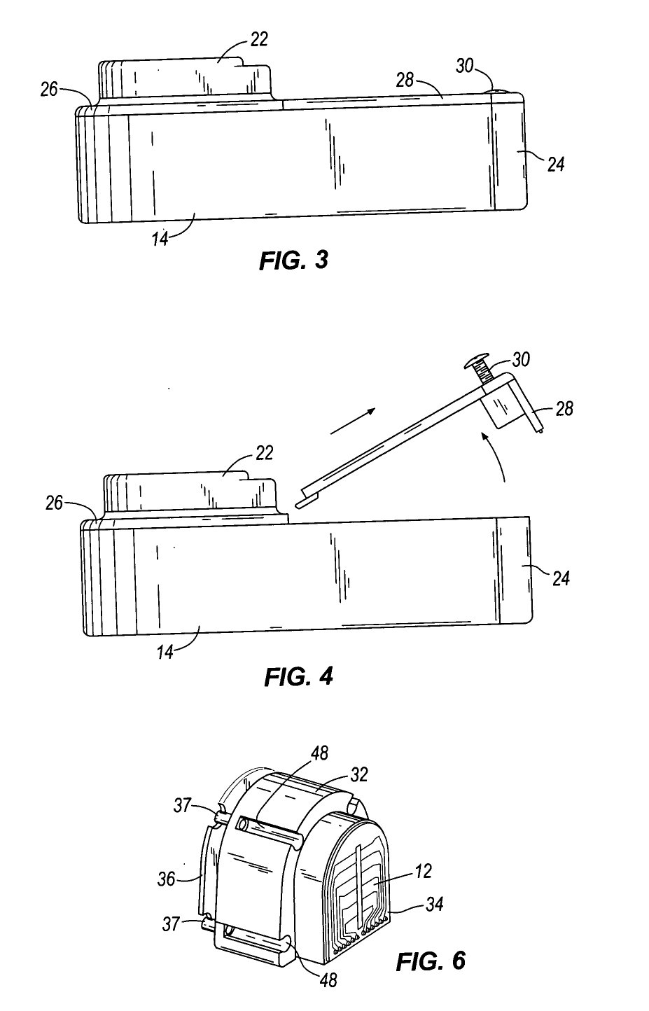

FIGS. 1 and 2 illustrate a hand-held device 10 according to one embodiment of the invention. As shown in FIG. 2, the hand-held device 10 includes one or more LEDs 12 (e.g., an array of LEDs) that can emit radiation toward a patient. The hand-held device 10 includes a housing 14 that supports the LEDs 12. The housing 14 can be constructed of a polycarbonate ABS alloy or any other suitable packaging polymer. In some embodiments, the housing 14 provides a sealed, self-contained enclosure for the hand-held device 10 so that no contaminates can enter the hand-held device 10. In other embodiments, the housing 14 includes vents so that air can pass through the housing 14 to cool the LEDs 12 or so that a fan (not shown) can be included in the housing 14 to cool the LEDs 12. If a fan is included in the housing 14, the hand-held device 10 can be powered by a portable power source within the housing 14 or by an AC power source (e.g., a power cord, a transformer, and/or an electrical plug for connection to a wall outlet). In some embodiments, a fan can provide continuous cooling, without a cooling-down period in which the LEDs 12 cannot be illuminated. A heat sink having fins (not shown) can also be used in conjunction with a fan to cool the LEDs 12.

As also shown in FIG. 2, the hand-held device 10 can include a cover plate 16 suitable to electrically isolate the patient from the LEDs 12. The cover plate 16 can be constructed of any suitable transparent or semi-transparent material. As shown in FIG. 1, the housing 14 can include one or more user-manipulatable controls 18 (e.g., a START button and a STOP button) and one or more indicator lights 20 (e.g., a LOW BATTERY light and a DELAY light).

As shown in FIG. 3, the housing 14 can include a raised portion 22 within which the LEDs 12 can be positioned. The raised portion 22 can include a circular aperture 23 (as shown in FIG. 2), or an aperture having any other suitable shape, through which the LEDs 12 can emit radiation. The cover plate 16 can be positioned within the raised portion 22 over the LEDs 12. The cover plate 16 can be coupled to the raised portion 22 with an ultraviolet epoxy or with any other suitable adhesive or fastener.

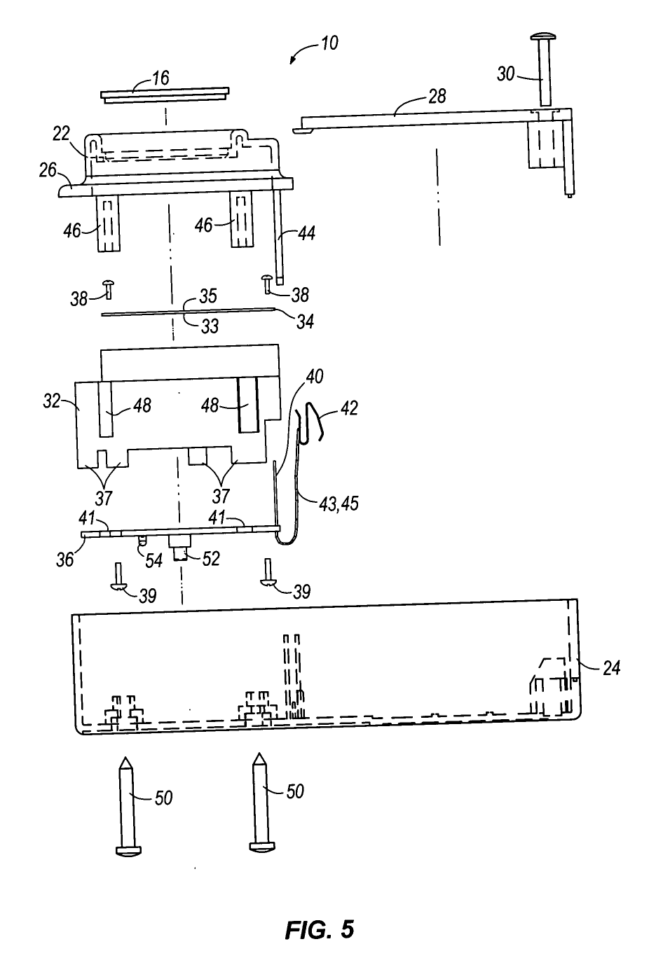

As shown in FIGS. 1 and 5, the housing 14 can include a top cover 24 and a bottom or aperture cover 26. The bottom cover 26 can include or can be coupled to the raised portion 22. As shown in FIGS. 4 and 5, the housing 14 can include a power source compartment cover 28 removably coupled adjacent to the bottom cover 26 with a screw 30. The hand-held device 10 can be powered by any suitable power source, including rechargeable or non-rechargeable, standard or non-standard batteries; AC power sources or connections; fuel cells; and other portable power sources. In one embodiment, the power source is eight standard AA-sized batteries which can be held together within the housing 14 by a battery holder.

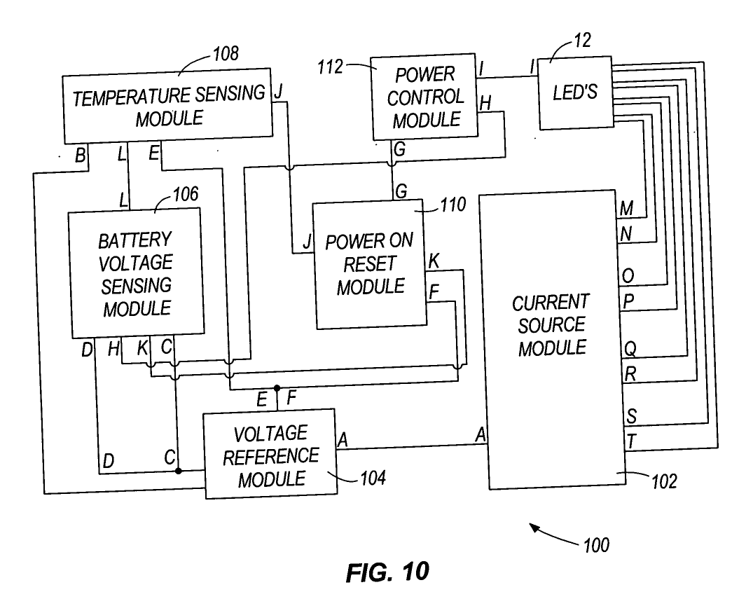

As shown in FIG. 5, the hand-held device 10 can include a cooling system in the form of a heat sink 32. The heat sink 32 can be constructed of aluminum, an aluminum alloy, or any other material suitable for dissipating heat. The heat sink 32 can have a total mass suitable for dissipating heat from the LEDs 12 during a cooling-down period of a reasonable duration (e.g., approximately 88 seconds after a single treatment session or several seconds longer after more than one consecutive treatment session). In one embodiment, the total mass of the heat sink 32 allows the hand-held device 10 to operate for eight to ten treatment sessions before the cooling-down period of 88 seconds must be extended (as will be described below with respect to FIGS. 10-16). The total mass of the heat sink 32 can also be designed so that the total weight of the hand-held device 10 (preferably including the batteries or other portable power source) is about one pound.

The heat sink 32 can be coupled to a first side 33 of a ceramic assembly 34 by one or more screws 38 (e.g., three nylon screws) or by a suitable thermal adhesive. The LEDs 12 (e.g., an array of several LEDs) can be coupled to a second side 35 of the ceramic assembly 34. The ceramic assembly 34 is thermally conductive in order to transfer heat emitted by the LEDs 12 to the heat sink 32, but the ceramic assembly 34 is not electrically conductive.

In other embodiments, the cooling system of the hand-held device 10 can include a thin-film insulator (not shown) coupled to an aluminum substrate (not shown). A suitable thin-film insulator is Kapton® manufactured by E. I. Du Pont De Nemours and Company Corporation.

As shown in FIG. 5, the heat sink 32 can also be coupled to a circuit board 36 by any suitable fasteners, such as screws 39 positioned through holes 41 in the circuit board 36. The heat sink 32 can include one or more elevated portions (or bosses or stand-offs) 37 that closely or directly contact one or more components mounted on the circuit board 36 (e.g., a temperature sensor and/or various transistors, as are described below with respect to FIGS. 10-16) in order to dissipate heat from those particular components. The elevated portions 37 can also create an air gap between the heat sink 32 and the circuit board 36 to further cool the components mounted on the circuit board 36. The elevated portions 37 can be integrally molded with the heat sink 32. The circuit board 36 can be connected to the LEDs 12 by a conductor jumper 40 (e.g., a twelve-conductor jumper in one embodiment or by two or more wires or groups or wires in other embodiments). The circuit board 36 can be connected to one or more batteries (not shown) or to any other suitable power source by a positive connection 43 (e.g., VBatt) and can be grounded with a ground wire 45 (as shown in FIG. 9). The positive connection 43 can be connected to one or more battery clips 42. The battery clips 42 can be attached to a partition wall 44 included in or coupled to the bottom cover 26. In some embodiments, when batteries are inserted into the housing 14, the battery clips 42 connect the positive ends of the batteries to the positive connection 43.

As also shown in FIG. 5, the bottom cover 26 can include one or more heat sink support members 46. The heat sink support members 46 can be positioned within corresponding recesses 48 on the edges of the heat sink 32. The top cover 24 of the housing 14, the bottom cover 26 of the housing 14, the heat sink 32, the ceramic assembly 34, and the circuit board 36 can be secured to one another by one or more suitable fasteners (e.g., screws 50), by suitable adhesives, or by a combination of fasteners and adhesives.

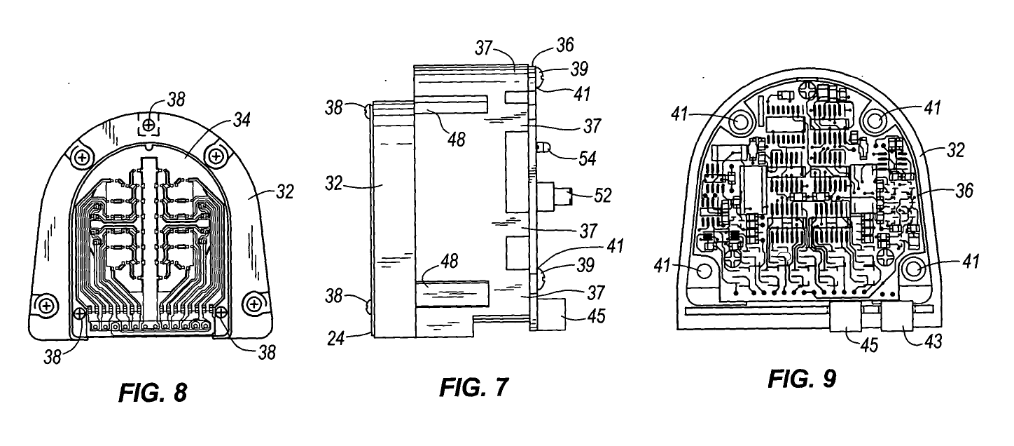

FIGS. 6 and 7 illustrate the LEDs 12, the heat sink 32, the ceramic assembly 34, and the circuit board 36 as assembled, but not positioned inside of the top cover 24 and the bottom cover 26 of the housing 14. FIG. 7 also illustrates a push button 52 coupled to the circuit board 36 (only one push button is shown from the side elevational view, although some embodiments include two push buttons for the two user-manipulatable controls 18 shown in FIG. 1). In addition, FIG. 7 illustrates an indicator light 54 (only one indicator light is shown from the side elevational view, although some embodiments include two indicator lights for the two indicator lights 20 shown in FIG. 1). FIG. 8 illustrates the LEDs 12 coupled to the ceramic assembly 34 and the heat sink 32. FIG. 9 illustrates the circuit board 36 coupled to the heat sink 32.

In some embodiments, the hand-held device 10 does not include a cooling system (i.e., no heat sink or fan). In these embodiments, the LEDs 12 are mounted to the circuit board 36 which is positioned inside of the housing 14. The LEDs 12 can be allowed to emit as much heat as possible without an additional cooling system.

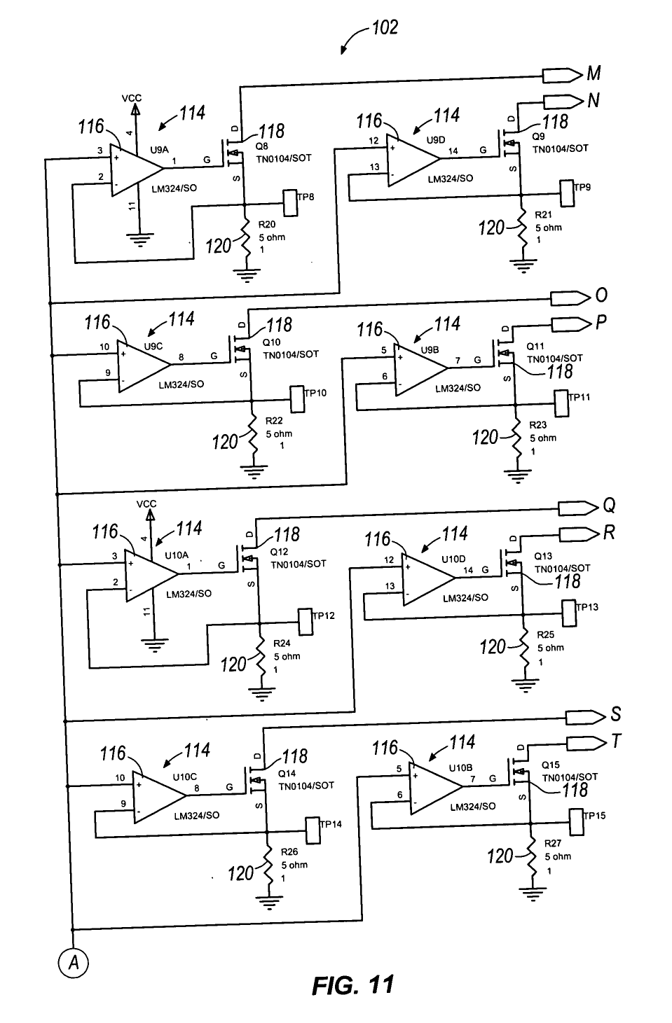

FIG. 10 is a schematic diagram of a control circuit 100 for use with the hand-held device 10. The components and connections of the control circuit 100 can be included in and/or mounted to the circuit board 36 described above. The control circuit 100 can include a current source module 102 that drives the LEDs 12 (via connections M through T). The current source module 102 can be connected to a voltage reference module 104 (via a connection A). The voltage reference module 104 can be connected to a battery voltage sensing module 106 (via connections C and D), a temperature sensing module 108 (via connections B and E), and a power-on reset module 110 (via a connection F). The power-on reset module 110 can be connected to a power control module 112 (via a connection G). The battery voltage sensing module 106 can be connected to the power control module 112 (via a connection H). The power control module 112 can be connected to the LEDs 12 (via a connection I). The temperature sensing module 108 can be connected to the power-on reset module 110 (via a connection J). The battery voltage sensing module 106 can be connected to the power-on reset module 110 (via a connection K) and to the temperature sensing module 108 (via a connection L). Particular embodiments of each of these modules will be described in detail with respect to FIGS. 11-16.

FIG. 11 illustrates one embodiment of the current source module 102. The current source module 102 can include eight current sources 114 resulting in eight channels being connected to the LEDs 12 (via connections M through T) in order to provide eight control signals or driving currents to the LEDs 12. In one embodiment, each channel is connected to six LEDs (e.g., two parallel strings of three LEDs in each string) for a total of 48 LEDs. In other embodiments, the LEDs 12 can be connected in any suitable manner, such as all of the LEDs being connected in series or all of the LEDs being connected in parallel, or any other combination of strings of LEDs being connected in series and in parallel. In some embodiments, any number of LEDs 12 can be connected in any manner as long as all of the LEDs can be turned ON and turned OFF at the same time. As shown in FIG. 10, each set of six LEDs can be connected to a positive power source V+(via the connection I) from the power control module 112. The current sources 114 can provide approximately 98 milliamps to the LEDs 12 connected to each one of the eight channels and approximately 49 milliamps to each string of three LEDs. Each one of the current sources 114 can include an operational amplifier 116. In one embodiment, two quad operational amplifiers can be used for the eight current sources 114 (a first quad operational amplifier includes U9A-U9D and a second quad operational amplifier includes U10A-U10D). Suitable operational amplifiers are Model No. LM324 operational amplifiers manufactured by National Semiconductor.

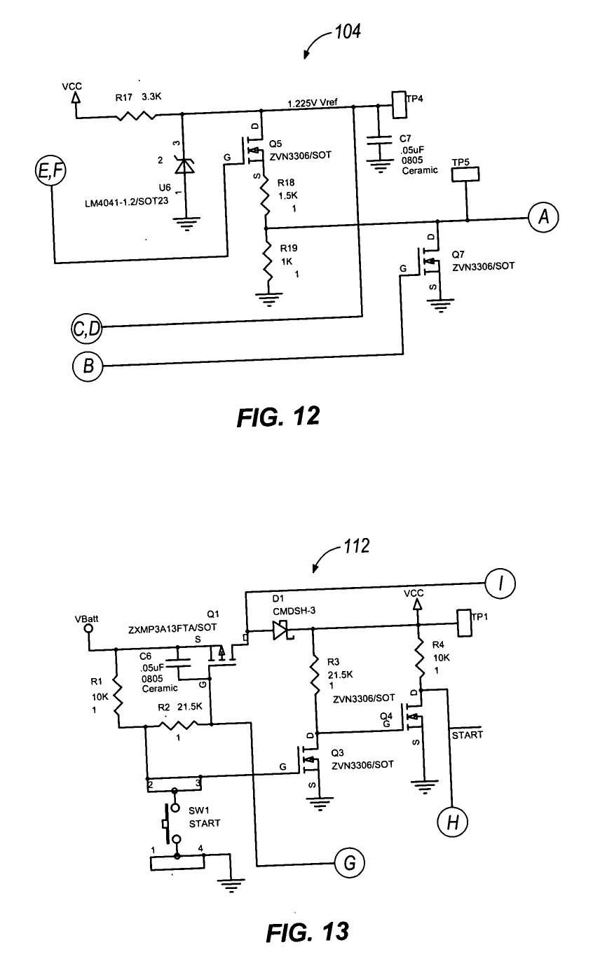

The output of each operational amplifier 116 can be connected to the gate of a transistor 118 (Q8-Q15). The drain of the transistor 118 can be connected to one set of six LEDs 12. Suitable transistors are Model No. TN0104 n-channel MOSFET transistors manufactured by Supertex. In each current source 114, a sensing resistor 120 (e.g., 5 Ohm resistors R20-R27) can be connected to a first input of the operational amplifier 116 and to the source of the transistor 118. The transistor 118 acts as a switch between the LEDs 12 and the positive power source V+ from the power control module 112. The sensing resistor 120 can determine how much current is being provided to the transistor 118 and the LEDs 12 at a test point (TP8-TP15). A second input of the operational amplifier 116 can be connected to a common node or test point TP5 in the voltage reference module 104 (at connection A as shown in FIG. 12).

Referring to FIGS. 11 and 12, the voltage at test point TP5 provides a reference voltage to each one of the current sources 114. In some embodiments, the test point TP5 reference voltage is approximately 0.49 Volts in order to provide 98 milliamps to each one of the current sources 114 (i.e., 98 milliamps to each set of six LEDs and 49 milliamps to each string of three LEDs). FIG. 12 illustrates one embodiment of the voltage reference module 104. Two resistors R18 (e.g., 1.5 kilo-ohms) and R19 (e.g., 1 kilo-ohm) can form a voltage divider circuit that provides the test point TP5 reference voltage. A voltage Vcc can be provided to a resistor R17 (e.g., 3.3 kilo-ohms) and to a diode U6 (e.g., a Model No. LM4041 zenar diode) for an output of 1.225 Volts (at test point TP4). A transistor Q5 (e.g., a Model No. ZVN3306 N-FET transistor manufactured by Zetex) can act as a switch to either provide 0.49 Volts (all the LEDs 12 are ON) or zero volts (all the LEDs are OFF) to test point TP5. A capacitor C7 (e.g., 0.05 microfarads) is a filtering and decoupling capacitor that can be connected to the drain of the transistor Q5.

As shown in FIG. 13, the power control module 112 can include three transistors Q1, Q3 and Q4. The transistor Q1 can be a Model No. ZXMP3A13 P-FET transistor manufactured by Zetex. The transistors Q3 and Q4 can be Model No. ZVN3306 N-FET transistors manufactured by Zetex. The power control module 112 can include a first tactile switch SW1 (e.g., a Model No. TL3301EF260QG or TL3301SPF260QG tactile switch manufactured by E-Switch). In one embodiment, a user can push the switch SW1 so that eight standard AA-sized batteries provide a battery voltage VBatt of 12 Volts to the control circuit 100. When a user presses the switch SW1, the gate of transistor Q1 is grounded and power can flow through the transistor Q1 (i.e., the transistor Q1 is turned ON). Thus, when a user presses the switch SW1, power from the batteries VBatt (or power from any other suitable power source) can flow through the transistor Q1 to the LEDs 12 via connection I. Power from the batteries VBatt can also flow through diode D1 (e.g., a Model No. CMDSH-3 Super Mini Schottky diode manufactured by Zetex) to provide a voltage Vcc at test point TP1. The diode D1 can provide reverse voltage protection from the batteries. The transistor Q3 can invert the signal from the transistor Q1 and can provide the inverted signal to the transistor Q4. The transistor Q4 can invert the signal again to generate a START signal (on the connection H). In some embodiments, once the transistors Q1, Q3 and Q4 are ON, the voltage Vcc can be 12 Volts. The power control module 112 can include resistors R1 (e.g., 10 kilo-ohms) and R2 (e.g., 21.5 kilo-ohms) connected between the battery voltage VBatt, the switch SW1, and the transistor Q1. The power control module 112 can also include a capacitor C6 (e.g., 0.05 microfarads) connected between the source and the gate of transistor Q1. In addition, the power control module 112 can include resistors R3 (e.g., 21.5 kiloohms) and R4 (e.g., 10 kilo-ohms) connected between the voltage Vcc and the drains of transistors Q3 and Q4, respectively.

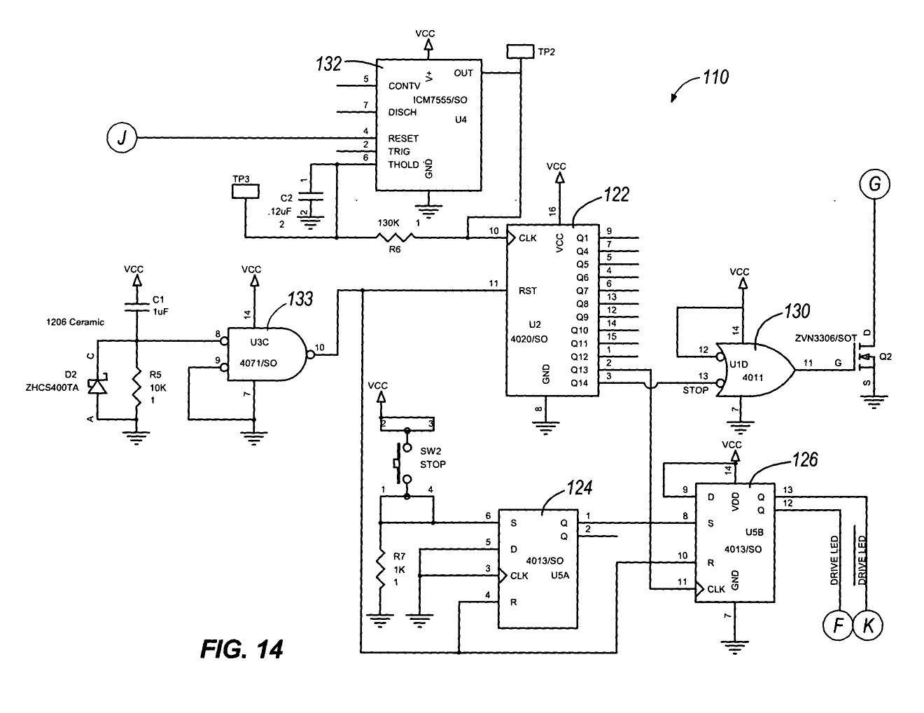

FIG. 14 illustrates one embodiment of the power-on reset module 110. The power-on reset module 110 can include a counter 122 (e.g., a Model No. CD4020 binary counter integrated circuit manufactured by Texas Instruments). The power-on reset module 110 can also include two flip-flops 124 and 126 (e.g., a Model No. CD4013 dual D-type flip-flop integrated circuit manufactured by Texas Instruments) connected to the counter 122. When the voltage Vcc is provided to the power-on reset module 110 after a user pushes the switch SW1, the counter 122 and the flip-flops 124 and 126 can be reset. When the voltage Vcc is provided to the power-on reset module 110, a pin Q14 of the counter 122 is initially at a zero state. The pin Q14 of the counter 122 can be connected to an inverter 130 (e.g., a Model No. CD4011 NAND gate manufactured by Texas Instruments). When the pin Q14 of the counter 122 provides a zero signal to the inverter 130, the output of the inverter 130 is a high signal, which turns a transistor Q2 ON (e.g., a Model No. ZVN3306 N-FET transistor manufactured by Zetex). The transistor Q2 of the power-on reset module 110 can be connected to the transistor Q1 of the power control module 112 (via the connection G). When the transistor Q2 is ON, the gate of the transistor Q Iis grounded and the transistor Q1 is ON.

The power-on reset module 110 can also include a 555 timer 132 (e.g., a Model No. ICM7555 general purpose 555 timer integrated circuit manufactured by Maxim and operating at a frequency of 45.8 Hz). Once a user turns the system ON by pressing the switch SW1, the 555 timer 132 can provide square waves or clock pulses to the counter 122 and to test point TP2. As the 555 timer 132 provides clock pulses, the counter 122 counts from pin Q1 to pin Q13, during which approximately 88 seconds can elapse. When pin Q13 goes to a high signal after 88 seconds, a clocking signal is provided to flip-flop 126, which then provides a DRIVE LED zero signal on pin 12 and a DRIVE LED high signal on pin 13 of the flip-flop 126. The DRIVE LED zero signal on pin 12 is provided to the transistor Q5 of the voltage reference module 104 (via the connection F) in order to turn the transistor Q5 OFF. When the transistor Q5 is OFF, the reference voltage at test point TP5 is zero and the LEDs 12 are OFF. The 555 timer 132 can continue to provide clock pulses until 88 more seconds (or any other suitable cooling-down period) have passed and pin Q14 of the counter 4020 provides a high signal. The high signal can be provided from pin Q14 of the counter 4020 to the inverter 130. The inverter 130 can provide a zero signal to turn OFF the transistor Q2, which also turns OFF the transistor Q1 of the power control module 112 (via the connection G) and turns OFF all power to the control circuit 100 (i.e., voltage Vcc is zero). In one embodiment, after the LEDs 12 are ON for a treatment session of 88 seconds, the LEDs are OFF for a cooling-down period of 88 seconds, and then all power is turned OFF to the control circuit 100.

The power-on reset module 110 can include a tactile switch SW2 (e.g., a Model No. TL3301EF260QG or TL3301SPF260QG tactile switch manufactured by E-Switch) that can be used as a STOP button. For example, if a user decides that he wants to turn the LEDs 12 OFF before the treatment session of 88 seconds has elapsed, the user can press the switch SW2. The switch SW2 is connected to the flip-flop 124 which is connected to the flip-flop 126. When the user presses the switch SW2, the flip-flop 126 provides a DRIVE LED zero signal on pin 12 which turns OFF the transistor Q5 of the voltage reference module 104. When the transistor Q5 is OFF, the reference voltage at test point TP5 is zero and the LEDs 12 are OFF.

The power-on reset module 110 can also include an AND gate 133, the output of which is connected to the counter 122. A capacitor C1 (e.g., 1 microfarads), a diode D2 (e.g., a Model No. ZHCS400TA diode), and a resistor R5 (e.g., 10 kilo-ohms) can be connected to one input of the AND gate 133. The other input of the AND gate 133 can be connected to ground. In addition, the power-on reset module 110 can include a capacitor C2 (e.g., 0.12 microfarads) connected to pins 2 and 6 of the 555 timer 132; a resistor R6 (e.g., 130 kilo-ohms) connected between pins 2 and 6 of the 555 timer 132 and a pin 10 of the counter 122; and a resistor R7 (e.g., 1 kilo-ohm) connected between the switch SW2 and a pin 6 of the flip-flop 124.

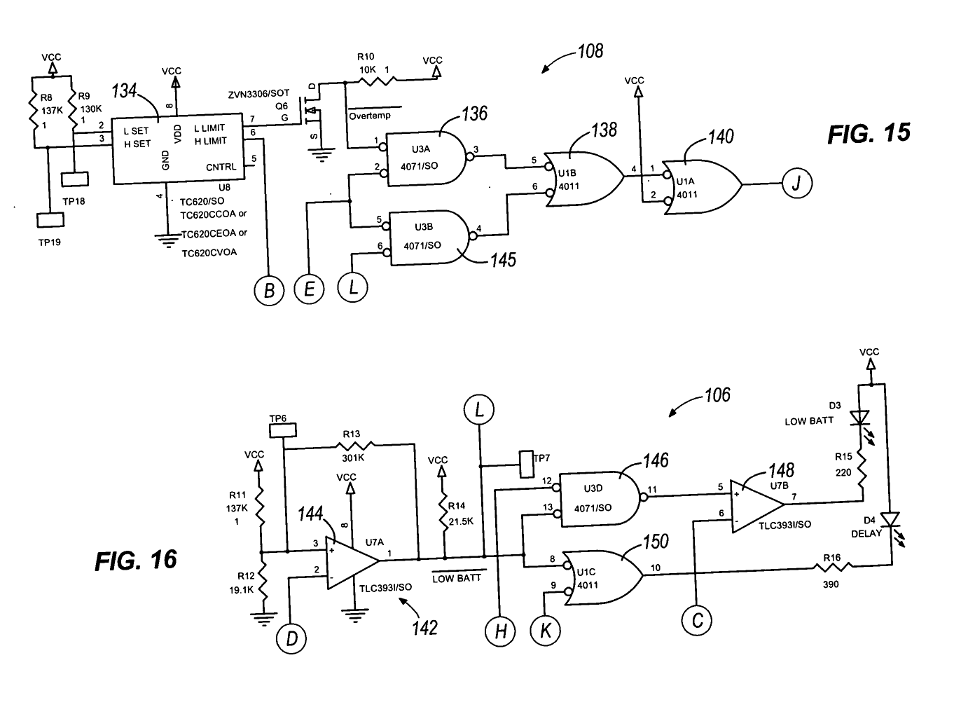

In some embodiments, as shown in FIG. 15, the control circuit 100 can include a temperature sensing module 108 that can be used to prevent the LEDs 12 from being turned ON if the heat emitted by the LEDs 12 has not been adequately dissipated. The temperature sensing module 108 can include a temperature sensor 134 (e.g., a Model No. TC620CVOA dual trip point temperature sensor integrated circuit manufactured by MicroChip). The temperature sensor 134 can have a low set point or first threshold temperature (e.g., 45.8 degrees C.) determined by resistor R9 (e.g., 130 kilo-ohms) and a high set point or a second threshold temperature (e.g., 53.8 degrees C.) determined by resistor R8 (e.g., 137 kilo-ohms). If the sensed temperature is greater than the high set point, the heat sink 32 and/or the LEDs 12 are too hot and, if the LEDs 12 are ON, the LEDs 12 can be turned OFF immediately. A pin 6 of the temperature sensor 134 is connected (via the connection B) to a transistor Q7 in the voltage reference module 104 (via the connection B). The transistor Q7 turns OFF the LEDs 12 when the sensed temperature exceeds the high set point (i.e., the reference voltage at test point TP5 becomes zero).

If the sensed temperature is greater than the low set point, but less than the high set point, the heat sink has not dissipated enough heat and the cooling-down period of the LEDs 12 can be extended. A pin 7 of the temperature sensor 134 can provide a high signal when the sensed temperature is greater than the low set point, but less than the high set point. The high signal can turn a transistor Q6 ON and can provide a zero signal to one input of an AND gate 136. A resistor R10 (e.g., 10 kilo-ohms) can be connected between the drain of the transistor Q6 and the voltage Vcc. A second input of the AND gate 136 can be connected to the pin 12 of the flip-flop 126 (via the connection B). The output signal of the AND gate 136 can be provided to a first inverter 138, which can provide an output signal to a second inverter 140. The second inverter 140 can be connected (via the connection J) to the 555 timer 132 of the power-on reset module 110. If the signal provided on the pin 12 of the flip-flop 126 indicates that the control circuit 100 has already turned the LEDs 12 ON for 88 seconds and the LEDs 12 are now OFF, but the sensed temperature is too high, the cooling-down period of the LEDs can be extended. The cooling-down period of the LEDs can be extended until the sensed temperature falls below the low set point. Once the sensed temperature falls below the low set point, a reset on the 555 timer 132 can be removed to allow the 555 timer 132 to finish providing clock pulses for an 88 second time period.

FIG. 16 illustrates one embodiment of the battery voltage sensing module 106. The battery voltage sensing module 106 can include a comparator circuit 142 that can determine whether the battery voltage is high enough to operate the control circuit 100 and the LEDs 12. The comparator circuit 142 can include a comparator 144 (e.g., a Model No. TLC393 dual comparator manufactured by Texas Instruments) and resistors R11 (e.g., 137 kilo-ohms), R12 (e.g., 19.1 kilo-ohms), and R13 (e.g., 301 kilo-ohms). A first input to the comparator 144 can be connected (via the connection D) to the reference voltage Vref (which can be 1.225 Volts) in the voltage reference module 104. A second input to the comparator 144 can be connected between resistors R11 and R12. If the comparator 144 determines that the voltage between resistors R11 and R12 is less than the reference voltage Vref, the output of the comparator 144 is a zero or low signal (LOW BATT) at test point TP7. A resistor R14 (e.g., 21.5 kilo-ohms) can be connected between the voltage Vcc and the output of the comparator 144. The output of the comparator 144 is also connected (via the connection L) to the first input of an AND gate 145 in the temperature sensing module 108. The second input of the AND gate 145 in the temperature sensing module 108 is connected (via the connection E) to the pin 12 of the flip-flop 126 of the power-on reset module 110 (which provides a DRIVE LED signal) and to the gate of the transistor Q5 of the voltage reference module 104. If the output of the comparator 144 is the LOW BATT signal, the temperature sensing module 108 (through the AND gate 145 and the inverters 138 and 140) can prevent the 555 timer 132 from restarting by holding the 555 timer 132 in a reset state. In some embodiments, when the 555 timer 132 cannot be restarted, the LEDs 12 cannot be turned ON when a user presses the START button.

The battery voltage sensing module 106 can also include a first diode D3 that can indicate to a user that the battery voltage is too low to operate the LEDs 12. The diode D3 can be connected to the comparator circuit 142 by an AND gate 146 and a comparator 148 (e.g., a Model TLC393 dual comparator manufactured by Texas Instruments). The inputs of the AND gate 146 can be connected to the output of the comparator 144 and to the drain of the transistor Q4 of the power control module 112 (via connection H). The inputs of the comparator 148 can be connected to the output of the AND gate 146 and the reference voltage Vref of the voltage reference module 104 (via connection C). The drain of the transistor Q4 of the power control module 112 can provide a START signal when a user presses the START button. Accordingly, when a user presses the START button and the comparator circuit 142 is providing the LOW BATT signal, the diode D3 lights up to indicate to the user that the LEDs will not turn ON due to the voltage of the batteries or the power source being too low.

The battery voltage sensing module 106 can include a second diode D4 that indicates to a user that the LEDs 12 will not turn ON during a cooling-down period. In some embodiments, after the LEDs 12 have been lit for 88 seconds, the cooling-down period can last another 88 seconds. The diode D4 can be connected to a resistor R16 (e.g., 390 Ohms) and an OR gate 150. The inputs of the OR gate 150 can be connected to the output of the comparator circuit 142 and to the flip-flop 126 of the power-on reset module 110 (via the connection K). Accordingly, when the comparator circuit 142 is providing the LOW BATT signal and the flip-flop 126 is providing a low or zero DRIVE LED signal, the diode D4 lights up to indicate to a user that the LEDs will not turn ON during the cooling-down period.

In some embodiments, the control circuit 100 can include one or more microprocessors in addition to or instead of the integrated circuits and individual electrical components described above with respect to FIGS. 10-16. A microprocessor can be programmed to perform any of the functions described above with respect to FIGS. 10-16 or any additional functions that are desired.

Rather than a cooling-down period having a fixed duration, in some embodiments, the control circuit 100 can increase the cooling-down period if not enough heat has been dissipated from the LEDs 12 or decrease the cooling-down period if enough heat has already been dissipated from the LEDs 12. The control circuit 100 can continually or intermittently monitor the temperature sensor 134 to determine when the temperature of the LEDs 12 and/or at least a portion of the circuit board 36 falls below a threshold temperature. In other embodiments, the control circuit 100 can be programmed to increase the cooling-down period after a certain number of treatment sessions and/or increase the cooling-down period after each consecutive treatment session. For example, after four 88 second treatment sessions, the control circuit 100 could extend the cooling-down period after the fourth treatment session to 100 seconds and the cooling-down period after the fifth treatment session to 120 seconds or greater. In some embodiments, the control circuit 100 includes a microprocessor programmed to increase or decrease the cooling-down period as described above.

According to the method of the invention, the hand-held device 10 can be positioned adjacent to the patient in a manner that allows the patient to absorb LED radiation. As one example, the hand-held device 10 can be positioned adjacent to the patient's leg. Once the hand-held device 10 is positioned in a manner that allows the patient to absorb LED radiation, the patient can be irradiated with LED radiation for treatment session having a predetermined time period, such as 88 seconds. In some embodiments, the patient is irradiated for 88 seconds at a power density of approximately 4 to 8 Joules per centimeter squared. However, the patient may be irradiated for shorter or longer periods of time at lesser or greater power densities. In some embodiments, the patient is irradiated for two or more treatment sessions of about 88 seconds each. A cooling-down period of about 88 seconds can be provided between treatment sessions, during which the LEDs are prevented from emitting radiation.

Although several embodiments of the present invention have been shown and described, alternate embodiments will be apparent to those skilled in the art and are within the intended scope of the present invention. Therefore, the invention is to be limited only by the following claims.

1. A method of treating at least one of muscle and joint pain being experienced by a patient, the method comprising:
positioning a housing adjacent to at least one of a muscle and ajoint of the patient, the housing including a plurality of optoelectronic devices;
irradiating the at least one of the muscle and the joint with radiation emitted by the plurality of optoelectronic devices, the emitted radiation having a wavelength suitable for the treatment of at least one of muscle and joint pain; and
dissipating heat produced by the plurality of optoelectronic devices.

2. The method of claim 1, and further comprising irradiating the at least one of the muscle and the joint with radiation at a wavelength of approximately 300 to 950 nanometers.

3. The method of claim 1, and further comprising irradiating the at least one of the muscle and the joint with radiation at a wavelength of approximately 640 to 700 nanometers.

4. The method of claim 1, and further comprising irradiating the at least one of the muscle and the joint with radiation at a wavelength of approximately 655 to 685 nanometers.

5. The method of claim 1, and further comprising irradiating the at least one of the muscle and the joint with radiation having an energy density of approximately 4 to 8 Joules per centimeter squared.

6. The method of claim 1, and further comprising irradiating the at least one of the muscle and the joint with radiation having a light intensity of approximately 30 to 80 milliwatts per centimeter squared.

7. The method of claim 1, and further comprising irradiating the at least one of the muscle and the joint at least once for approximately 80 to 100 seconds to treat at least one of muscle and joint pain.

8. The method of claim 1, and further comprising positioning the housing near skin adjacent to the at least one of the muscle and the joint.

9. A self-contained, self-powered, hand-held device for treating at least one of muscle and joint pain being experienced by a patient, the device comprising:
a housing;
a portable power source disposed in the housing;
at least one optoelectronic device disposed in the housing and coupled to the portable power source, the at least one optoelectronic device emitting radiation having a light intensity of at least approximately 30 milliwatts per centimeter squared; and
a cooling system disposed in the housing, the cooling system dissipating heat generated by the at least one optoelectronic device.

10. The device of claim 9, wherein the at least one optoelectronic device includes an array of light-emitting diodes.

11. The device of claim 9, wherein the at least one optoelectronic device emits radiation at a wavelength of approximately 300 to 950 nanometers.

12. The device of claim 9, wherein the at least one optoelectronic device emits radiation at a wavelength of approximately 640 to 700 nanometers.

13. The device of claim 9, wherein the at least one optoelectronic device emits radiation at a wavelength of approximately 655 to 685 nanometers.

14. The device of claim 9, wherein the at least one optoelectronic device emits radiation having an energy density of approximately 4 to 8 Joules per centimeter squared.

15. The device of claim 9, wherein the at least one optoelectronic device emits radiation having a light intensity of approximately 30 to 80 milliwatts per centimeter squared.

16. The device of claim 9, wherein the at least one optoelectronic device emits radiation having a light intensity of approximately 50 milliwatts per centimeter squared.

17. The device of claim 9, wherein the housing is positioned adjacent to at least one of a muscle and a joint of the patient and the at least one optoelectronic device emits radiation toward the patient for a treatment session of approximately 80 to 100 seconds.

18. The device of claim 9, and further comprising a cover plate coupled to the housing to electrically isolate the patient from the at least one optoelectronic device.

19. The device of claim 9, wherein the cooling system includes a heat sink disposed in the housing.

20. The device of claim 19, wherein the heat sink is constructed substantially of an aluminum alloy.

21. The device of claim 19, wherein the plurality of optoelectronic devices are coupled to a circuit board and the circuit board is coupled to the heat sink.

22. The device of claim 19, wherein the housing does not include a vent.

23. The device of claim 9, wherein the cooling system includes a fan and the housing includes at least one vent.

24. The device of claim 9, wherein the cooling system includes a temperature sensor and a control circuit, wherein the control circuit is coupled to the temperature sensor and to the at least one optoelectronic device, and wherein the control circuit interrupts power to the at least one optoelectronic device based on a temperature sensed by the temperature sensor.

25. The device of claim 24, wherein the control circuit alters a cooling-down period between two treatment sessions so that heat is adequately dissipated from the at least one optoelectronic device before a new treatment session can be started.

26. The device of claim 24, wherein the control circuit prevents the at least one optoelectronic device from operating until a sensed temperature of the device is less than a threshold temperature.

27. The device of claim 26, wherein the threshold temperature is approximately 53 to 54 degrees Celsius.

28. The device of claim 9, wherein the portable power source includes at least one standard AA-sized battery.

29. The device of claim 9, wherein the housing includes an array of light-emitting diodes, the array having a diameter of approximately three centimeters, and the array including up to approximately 48 light-emitting diodes.

30. The device of claim 9, wherein the at least one optoelectronic device includes approximately four to five light-emitting diodes per centimeter squared.

31. The device of claim 9, and further comprising a control circuit that allows the at least one optoelectronic device to emit radiation for a treatment session of approximately 80 to 100 seconds and then prevents the at least one optoelectronic device from emitting radiation for a cooling-down period of at least about 80 seconds.

32. A method of treating at least one of muscle and joint pain being experienced by a patient, the method comprising:
positioning a housing adjacent to at least one of a muscle and a joint of the patient, a plurality of optoelectronic devices being disposed in the housing;
irradiating the at least one of the muscle and the joint with radiation emitted by the plurality of optoelectronic devices for a treatment session having a first duration;
allowing the plurality of optoelectronic devices to dissipate heat for a cooling-down period having a second duration; and
preventing the plurality of optoelectronic devices from emitting radiation during the cooling-down period.

33. The method of claim 32, and further comprising irradiating the at least one of the muscle and the joint for a treatment session having a first duration of approximately 80 to 100 seconds.

34. The method of claim 32, and further comprising allowing the plurality of optoelectronic devices to dissipate heat for a cooling-down period having a second duration of at least about 80 seconds.

35. The method of claim 32, and further comprising sensing a temperature of at least one of the plurality of optoelectronic devices.

36. The method of claim 35, and further comprising increasing the second duration of the cooling-down period if the sensed temperature is greater than a first threshold temperature.

37. The method of claim 36, and further comprising turning the plurality of optoelectronic devices off if the sensed temperature is greater than a second threshold temperature that is higher than the first threshold temperature.

38. The method of claim 32, and further comprising indicating to a user that the plurality of optoelectronic devices will not emit radiation during the cooling-down period.

39. The method of claim 32, and further comprising irradiating the at least one of the muscle and the joint for a treatment session having a first duration equal to the energy density of the emitted radiation divided by the light intensity of the emitted radiation.

40. The method of claim 32, and further comprising irradiating the at least one of the muscle and the joint with radiation at a wavelength of approximately 300 to 950 nanometers.

41. The method of claim 32, and further comprising irradiating the at least one of the muscle and the joint with radiation at a wavelength of approximately 640 to 700 nanometers.

42. The method of claim 32, and further comprising irradiating the at least one of the muscle and the joint with radiation at a wavelength of approximately 655 to 685 nanometers.

43. The method of claim 32, and further comprising irradiating the at least one of the muscle and the joint with radiation having an energy density of approximately 4 to 8 Joules per centimeter squared.

44. The method of claim 32, and further comprising irradiating the at least one of the muscle and the joint with radiation having a light intensity of approximately 30 to 80 milliwatts per centimeter squared.

45. The method of claim 32, and further comprising positioning the housing near skin adjacent to the at least one of the muscle and the joint.
